(19) United States
(12) Patent Application Publication
(10) Pub. No.: US 2004/0265247 A1
(43) Pub. Date: Dec. 30, 2004

(54) Mouth rinse composition

(75) Inventors: Masao Abiru, Tokyo (JP); Takuya Sato, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: GC Corporation, Tokyo (JP)

(21) Appl. No.: 10/847,311

(22) Filed: May 18, 2004

(30) Foreign Application Priority Data
May 20, 2003 (JP) 2003-141422

(51) Int. Cl. A61K 7/20; A61K 35/78

(52) U.S. Cl. 424/58; 424/771

(57) ABSTRACT

A mouth rinse composition that is safe even if accidentally ingested, can be easily used by aged persons and children without uncomfortable feeling, and is clearly distinguished from the conventional mouth rinse compositions, comprises a combination of an extract of a persimmon or persimmon tannin and an extract of grapefruit seed, which effectively sterilize wide range of offending bacteria causing deterioration in intra-oral environment, such as dental caries, periodontal disease, oral odor and coated tongue, present in an oral, and an algefacient and/or an edulcorant gentle to teeth, which makes the composition easy to use for aged persons and children.

### BACKGROUND OF THE INVENTION

1. Field of the Invention

The present invention relates to a mouth rinse composition capable of obtaining bactericidal effect to various bacteria present in an oral cavity in an effective and safe manner.

2. Description of the Related Art

Examples of a mouth rinse composition having been used as a detergent for an oral using chemical medicinal effect include those containing povidone iodine, domiphen bromide, benzethonium chloride, fradiomycin sulfate, sodium azulene sulfonate or the like dissolved in a solvent, such as water and ethanol.

Under consideration of consumers of a mouth rinse composition as a detergent for an oral, there are different demands depending on the segment of consumers such as adults, children or aged persons. Specifically, there is generally no severe problem in the case where the aforementioned mouth rinse compositions are used for eliminating oral odor by removing bacteria and viruses in an oral. However, there are some cases of bedridden aged persons suffering from deterioration of conditions in the oral, or having difficulty in detergence itself of the oral. In these cases, there is a high risk to an impaired human body, as the aforementioned mouth rinse composition using chemical medicinal effect may be accidentally ingested when deterging an oral cavity. The safety to a human body of the mouth rinse composition using chemical medicinal effect is ensured under such an assumption that the mouth rinse composition is spit after deterging, but it does not assumes accidental ingestion. It is apparent that the composition causes some maleficence to a body of an impaired bedridden aged person upon accidental ingestion.

The mouth rinse compositions containing natural substances or materials extracted from natural substances have been traditionally used. Those mouth rinse compositions have been empirically recognized by human kind as having desired effects and extracts exhibiting higher effect are produced from them by current advanced production techniques to be effectively utilized for sterilization of an oral. For example, extracts of tea, such as green tea and oolong tea, and Mentha family plants are disclosed, for example, in JP-A-2000-128801 and JP-A-6-56687, products wherein grapefruit and seed thereof are used as an antibacterial (sterilizing) component are disclosed, for example, in JP-A-9-208787 and International Application 00/11956, while they are not used for an oral, and extracts of rosemary, Urajirogashi (Quercus salicina), mustard, bamboo and Japanese plum are disclosed, for example, in JP-A-11-304312. These materials are advantageous owing to high safety thereof since they are diet-induced materials. Many kinds of products are commercially available with wide range of varieties by utilizing the advantages of these materials.

The mouth rinse compositions containing natural substances and extracts from natural substances have been empirically recognized by human kind to have effects as they are ingested from foods, and safety of most of them has been empirically ascertained. However, natural substances and extracts thereof have strong bitter flavor, and children are sensitive in gustation. Therefore, the mouth rinse compositions containing the natural substances or extracts thereof can exert the expected effects for ordinary adults, who can bear the bitter flavor, but are not currently utilized for children.

### SUMMARY OF THE INVENTION

An object of the present invention is to provide such a mouth rinse composition that is safe even in the case of accidental ingestion and has less bitter flavor, and that therefore can be easily used by aged persons and children without uncomfortable feeling.

As a result of earnest investigations made by the inventors for solving the problems, it has been found that a combination of an extract of a persimmon or persimmon tannin and an extract of grapefruit seed can effectively sterilize offending bacteria present in an oral cavity, which significantly deteriorate intra-oral environments to cause, for example, dental caries, periodontal disease, oral odor and coated tongue. It has been also found that a mouth rinse composition formed by adding an algefacient and/or an edulcorant gentle to teeth to water in a certain proportion along with the aforementioned combination of an extract of a persimmon or persimmon tannin and an extract of grapefruit seed is gentle to aged persons and children. Thus a mouth rinse composition having clear distinction from the conventional mouth rinse compositions has been made.

The present invention relates to a mouth rinse composition containing water, an extract of a persimmon or persimmon tannin, an extract of grapefruit seed, and an algefacient and/or an edulcorant.

### DETAILED DESCRIPTION OF THE INVENTION

The reason for use of the combination of an extract of a persimmon or persimmon tannin and an extract of grapefruit seed is described as follows. An extract of a persimmon or persimmon tannin itself not only has antibacterial (sterilizing) effect of sterilizing mutans bacteria, which are offending bacteria of dental caries, but also has the following effect. An extract of a persimmon or persimmon tannin has been often used when producing refined sake in sake factories for removing impurity proteins since it is specifically combined with the proteins during production of refined sake. In an oral, dental plaque contains polysaccharides derived from foods along with mutans bacteria. The polysaccharides include dextran and levan, which are formed from glucose and fructose, respectively. The polysaccharides are decomposed by the mutans bacteria to form acids, which corrodes enamel of teeth to cause dental caries. The extract of a persimmon or persimmon tannin is significantly excellent in effect of suppressing formation of dental plaque because it quickly forms strong combination with proteins in comparison to other catechin compounds. This is because the main component of the extract of a persimmon or persimmon tannin is a condensed catechin compound, which is a proanthocyanidin polymer having a molecular weight of about 15,000 having four repeating structures of epicatechin, catechin-3-gallate, epigallocatechin and gallocatechin-3-gallate, and has such an effect that suppresses growth of mutans bacteria. The extract of grapefruit seed cannot sufficiently sterilize bacteria present inside dental plaque only by the antibacterial (sterilizing) power thereof to cause such a severe problem that bacteria present in dental plaque again propagate with the lapse of time. Persimmon catechin quickly forms strong combination with proteins of dental plaque, and therefore, the mouth rinse composition of the present invention solves the severe problem by using the extract of a persimmon or persimmon tannin and the extract of grapefruit seed in combination.

The mouth rinse composition of the present invention contains, as a mixture, the extract of a persimmon or persimmon tannin, which is commercially available as Pancil BA (a trade name, produced by Release Scientific Industries, Ltd.), and the extract of grapefruit seed, which is commercially available as Desfan-100 (a trade name, imported and distributed by Mitsuba Corp.). The weight ratio of the extract of a persimmon or persimmon tannin and the extract of grapefruit seed is preferably 3/7 to 7/3. The mixture is preferably contained in the mouth rinse composition in a proportion of 0.05 to 5% by weight.

The extract of a persimmon or persimmon tannin sterilizes wide variety of bacteria and has MIC (minimum inhibitory concentration) in ppm order to gram-positive bacteria, gram-negative bacteria, fungi and molds. It is considered that the effect is attained mainly by a phenol effect of a flavonoid compound and an electrostatic effect of a fatty acid. It is considered that the extract of grapefruit seed has such an effect that is equivalent to polylysine, a middle chain glycerin fatty acid ester and a sucrose fatty acid ester. The extract of grapefruit seed has not only effects on general bacteria, fungi, Pseudomonas aeruginosa and cariogenic Streptococcus, but also effects for elimination of oral odor, and therefore, it is effective to wide variety of fields in dental clinical.

The mouth rinse composition of the present invention is prepared to have some exhilaration and sweet taste for facilitating deterging of oral of children and aged persons without uncomfortable feeling. In the case where only the mixture of the extract of a persimmon or persimmon tannin and the extract of grapefruit seed is mixed with water to form a mouth rinse composition, there is such a possibility that children cannot bear bitter flavor and astringent taste inherent in the extract of a persimmon or persimmon tannin and the extract of grapefruit seed, aside from ordinary adults, and therefore, an edulcorant is mixed with the extract of a persimmon or persimmon tannin and the extract of grapefruit seed. Examples of effective edulcorant include stevia, xylitol, sorbitol, oligosaccharide, acesulfame potassium, trehalose and aspartame. Sugar is not suitable as the edulcorant in this case because it is decomposed in an oral, and lactic acid thus produced causes decalcification of enamel of teeth to cause dental caries. The mixing proportion of the edulcorant widely varies depending on the degree of sweetness thereof, and in general, it is preferably mixed in an amount of 0.01 to 5% by weight based on the total mouth rinse composition. For example, stevia having a degree of sweetness 200 times that of sugar may be mixed in an amount of 0.01 to 0.1% by weight based on the total mouth rinse composition.

The algefacient is mixed in the mouth rinse composition of the present invention to obtain exhilaration, and various kinds of herbs can be used. In particular, peppermint, menthol and mint are preferably used. The algefacient is suitably mixed in an amount of 0.05 to 0.5% by weight based on the total mouth rinse composition. It has been confirmed that some kinds of the edulcorant and the algefacient themselves, for example, xylitol, stevia, herbs and mint, have an effect of preventing dental caries.

The mouth rinse composition of the present invention contains the combination of natural substances, i.e., the extract of a persimmon or persimmon tannin and the extract of grapefruit seed, whereby it can effectively sterilize wide range of offending bacteria causing deterioration in intra-oral environment, such as dental caries, periodontal disease, oral odor and coated tongue, present in an oral. At the same time, an algefacient and/or an edulcorant gentle to teeth are contained in the mouth rinse composition together with the extract of a persimmon or persimmon tannin and the extract of grapefruit seed for facilitating deterging of oral of children and aged persons without uncomfortable feeling, whereby such a mouth rinse composition can be provided that is clearly distinguished from the conventional mouth rinse compositions for ordinary adults. It is apparent to a skilled person in the art that the mouth rinse composition of the present invention may contain a solvent, such as ethanol and glycerin, a coloring agent, a preservative and the like in such an amount that does not impair the capabilities of the aforementioned effective components.

It is also apparent to a skilled person in the art that the combination of the extract of a persimmon or persimmon tannin and the extract of grapefruit seed, which is a part of the mouth rinse composition of the present invention, may be added to an aqueous solution component of conventional dental cement and a conventional dental impression material to utilize it for effectively sterilizing wide range of offending bacteria causing deterioration in intra-oral environment, such as dental caries, periodontal disease, oral odor and coated tongue, present in an oral. It is further apparent that it is possible that the extract of a persimmon or persimmon tannin and the extract of grapefruit seed are formed into a powder form by utilizing the clathrate function of dextrin, and mixed with a dental composite resin in a suitable amount, whereby a persistent antibacterial effect is obtained by extended and controlled release thereof.

### EXAMPLE

The present invention will be described in more detail with reference to the following examples, but the present invention is not construed as being limited thereto.

The mouth rinse compositions were produced in the following manners.

### Example 1

0.3% by weight of spearmint and 0.5% by weight of a mixture of an extract of a persimmon tannin and an extract of grapefruit seed in a ratio of 1/1 were mixed with 99.2% by weight of distilled water to obtain a mouth rinse composition.

### Example 2

0.07% by weight of stevia and 3% by weight of a mixture of an extract of persimmon tannin and an extract of grapefruit seed in a ratio of 1/1 were mixed with 96.93% by weight of distilled water to obtain a mouth rinse composition.

### Example 3

0.02% by weight of stevia, 0.1% by weight of spearmint and 0.1% by weight of a mixture of an extract of persimmon tannin and an extract of grapefruit seed in a ratio of 3/7 were mixed with 99.78% by weight of distilled water to obtain a mouth rinse composition.

### Example 4

0.07% by weight of stevia and 0.5% by weight of a mixture of an extract of a persimmon and an extract of grapefruit seed in a ratio of 3/7 were mixed with 99.43% by weight of distilled water to obtain a mouth rinse composition.

### Example 5

0.07% by weight of stevia and 3% by weight of a mixture of an extract of persimmon tannin and an extract of grapefruit seed in a ratio of 3/7 were mixed with 96.93% by weight of distilled water to obtain a mouth rinse composition.

### Example 6

0.02% by weight of stevia, 0.1% by weight of spearmint and 0.1% by weight of a mixture of an extract of persimmon tannin and an extract of grapefruit seed in a ratio of 7/3 were mixed with 99.78% by weight of distilled water to obtain a mouth rinse composition.

### Example 7

0.07% by weight of stevia and 0.5% by weight of a mixture of an extract of a persimmon and an extract of grapefruit seed in a ratio of 7/3 were mixed with 99.43% by weight of distilled water to obtain a mouth rinse composition.

### Example 8

0.3% by weight of spearmint and 3% by weight of a mixture of an extract of persimmon tannin and an extract of grapefruit seed in a ratio of 7/3 were mixed with 96.7% by weight of distilled water to obtain a mouth rinse composition.

### Comparative Example 1

Sterilized distilled water was prepared.

### Comparative Example 2

0.3% by weight of stevia and 0.1% by weight of spearmint were mixed with 99.9% by weight of distilled water to obtain a mouth rinse composition.

### Comparative Example 3

0.5% by weight of a mixture of an extract of persimmon tannin and an extract of grapefruit seed in a ratio of 1/1 was mixed with 99.5% by weight of distilled water to obtain a mouth rinse composition.

### Antibacterial Activity Test

The mouth rinse compositions of Examples 1 to 8 and Comparative Examples 2 and 3 were subjected to an antibacterial activity test in the following manner.

100 μL each of glycerol stocks of S. Mutans ATCC25175 strain and S. Sobrinus ATCC33475 strain each was planted to about 20 mL of a BHI liquid culture medium, and subjected to static culture at 37° C. overnight. A suspension of S. Mutans ATCC25175 strain and a suspension of S. Sobrinus ATCC33475 each having a concentration of 2×10⁷ cells per mL were prepared, and 100 μL of each of them were smeared on an MSB agar culture medium. Two pieces of filter paper having a diameter of 8 mm were placed on the culture medium having the suspension smeared thereon, and the control (Comparative Example 1) and the mouth rinse composition of Example 1 each was dropped thereon in an amount of 50 μL, respectively. The combination of the control and Example 1 is referred to as a set for Example 1. Sets for Examples 2 to 8 and Comparative Examples 2 and 3 were prepared in the same manner as in the set for Example 1, i.e., the control (Comparative Example 1) and the mouth rinse composition of one of Examples and Comparative Examples was dropped on the pieces of filter paper in an amount of 50 μL, respectively.

All the sets were cultured at 37° C. for 2 days, and the antibacterial activity of Examples and Comparative Examples was evaluated by the size of the inhibition circles. The results obtained are shown in Table 1 below. All the compositions exhibited the similar tendency in antibacterial activity to both S. Mutans ATCC25175 strain and S. Sobrinus ATCC33475 strain evaluated from the size of the inhibition circles, and therefore, the evaluation was made based on the following standard.

A: A large inhibition circle was obtained in comparison to the control (Comparative Example 1) to exhibit significant antibacterial activity.

B: A certain inhibition circle was obtained in comparison to control (Comparative Example 1) to exhibit certain antibacterial activity.

C: Substantially no inhibition circle was obtained, and no antibacterial activity could be expected.

### Gustation Test

Uncomfortable feeling to bitter flavor and astringent taste of the extract of persimmon or persimmon tannin and the extract of grapefruit seed, in particular, the degree of feeling bitter flavor and astringent taste, was evaluated by the following manner.

Subjects including one female of 20's, one male and one female of 30's, one male of 40's and one male of 50's were randomly selected. Subjects held in their oral cavities the mouth rinse compositions of Examples 1 to 8 and Comparative Examples 2 and 3 in random sequence to evaluate uncomfortable feeling, and then the oral were deterged with distilled water to evaluate uncomfortable feeling to bitter flavor and astringent taste of the mouth rinse compositions. The results obtained are shown in Table 1 below.

The evaluation of the uncomfortable feeling to bitter flavor and astringent taste for each of Examples and Comparative Examples was made by the respective subjects selectively applying points of the following standard.

0 point: No uncomfortable feeling to bitter flavor and astringent taste was caused.

1 point: Slight uncomfortable feeling to bitter flavor and astringent taste was caused.

3 points: Significant uncomfortable feeling to bitter flavor and astringent taste was caused.

In the case where the total points applied by all the subjects was 0 to 2 points, it was evaluated as A in the gustation test. In the case where the total points was 3 to 7 points, it was evaluated as B, and in the case where the total points was 8 to 12, it was evaluated as C.

TABLE 1

| | | | | | | | | | Comparative | |
|---|---|---|---|---|---|---|---|---|---|---|
| | Example | | | | | | | | Example | |
| | 1 | 2 | 3 | 4 | 5 | 6 | 7 | 8 | 2 | 3 |
| Antibacterial | B | B | A | A | B | B | A | A | C | A |
| activity test | | | | | | | | | | |
| Gustation | A | B | B | A | A | A | A | B | A | C |
| test | | | | | | | | | | |

As is confirmed by the results of the aforementioned Examples, the mouth rinse composition according to the present invention effectively sterilizes offending bacteria present in an oral cavity by the combination of natural substances, i.e., an extract of a persimmon or persimmon tannin and an extract of grapefruit seed, and can be used for deterging oral of children and aged persons without uncomfortable feeling by containing an algefacient and/or an edulcorant gentle to teeth along with the combination of an extract of a persimmon or persimmon tannin and an extract of grapefruit seed. Consequently, such a mouth rinse composition is obtained that is gentle to aged persons and children and is clearly distinguished from the conventional mouth rinse compositions.

What is claimed is:

1. A mouth rinse composition comprising water, an extract of a persimmon or persimmon tannin, an extract of grapefruit seed, and an algefacient and/or an edulcorant.
